(19) United States
(12) Patent Application Publication
(10) Pub. No.: US 2025/0223254 A1
(43) Pub. Date: Jul. 10, 2025

(54) METHOD FOR ROOM TEMPERATURE DEPOLYMERIZATION OF TEREPHTHALIC POLYESTERS TO TEREPHTHALATE ESTERS

(71) Applicant: RECYC'ELIT, Venissieux (FR)

(72) Inventors: Ayoub El Mahdi, Vienne (FR); Raouf Medimagh, Lyon (FR); Théo Fourdin, Lyon (FR)

(21) Appl. No.: 18/854,306

(22) PCT Filed: Apr. 5, 2023
(86) PCT No.: PCT/EP2023/058949

§ 371 Date: Oct. 4, 2024

(30) Foreign Application Priority Data
Apr. 6, 2022 (FR) FR2203148
Jan. 19, 2023 (FR) FR2300523

(51) Int. Cl. C07C 69/82

(52) U.S. Cl. CPC C07C 69/82

(57) ABSTRACT

A method for recycling a material comprising terephthalate polyester into terephthalate diester includes milling or shredding the material to produce fragments, and depolymerizing the polyester into terephthalate ester in the presence of: (i) a catalyst selected from a metal or organic ether oxide base, a metal acetate, a metal oxide, a metal hydroxide, a metal ester or a metal carbonate, (ii) a polar solvent of the cyclic ester or ether oxide type, and (iii) an alcohol selected from a monoalcohol or a diol. The base is present in catalytic amount relative to the amount of the polyester, and the depolymerization step is carried out at ambient temperature or by heating up to 70° C. for a period of between 1 minute and 4 hours.

### CROSS-REFERENCE TO RELATED APPLICATIONS

This application is a national phase entry under 35 U.S.C. § 371 of International Patent Application PCT/EP2023/058949, filed Apr. 5, 2023 designating the United States of America and published as International Patent Publication WO 2023/194442 A1 on Oct. 12, 2023, which claims the benefit under Article 8 of the Patent Cooperation Treaty of French Patent Application Serial No. FR2203148, filed Apr. 6, 2022 and of Patent Cooperation Treaty of French Patent Application Serial No. FR2300523, filed Jan. 19, 2023.

### TECHNICAL FIELD

The present disclosure relates to the field of recycling materials comprising terephthalate polyester, in particular, polyethylene terephthalate (PET) or polybutylene terephthalate (PBT) commonly used for manufacturing disposable plastic bottles, food trays, fabrics, composite material for insulation, etc. It relates, in particular, to a method for recycling PET into terephthalic diester and, in particular, dimethyl terephthalate (DMT) in less than one hour and without a pretreatment step. In addition, this method does not use any toxic products. It is therefore particularly advantageous from an industrial point of view.

### BACKGROUND

PET recycling is a major environmental matter and thus represents a commercial opportunity due to its generalized use, its abundance and its durability. However, the recycling of PET-based materials is complex and varies according to the type of polymer, the design of the material and the type of finished product.

The main impediment for the use of recycled plastic materials is the contamination of waste streams with different types of polymers that are not compatible with one another. As a result, it is often not possible to add recycled PET-type plastic to virgin polymer without reducing certain quality attributes, such as color, clarity or impact resistance. For this reason, the ability to replace a virgin polymer with recycled PET depends greatly on the purity of the recycled product and the requirements of the final product.

According to the principle of chemical recycling, PET can be depolymerized by a solvolysis such as methanolysis or glycolysis, or by hydrolysis, and the monomers thus obtained can be reused to generate new PET polymers referred to as “recycled PET.”

Depending on industrial needs, certain technologies for manufacturing PET resin resort to the use of terephthalic acid dimethyl ester (DMT).

In addition, conventional methanolysis techniques use methods that are very energy intensive and costly in equipment. These methods use a supercritical phase at temperatures higher than 300° C. and at pressures between 5 and 10 bar. Due to drastic reaction conditions in terms of temperatures and pressure, these technologies induce structural changes to the molecular units of the PET, in particular, an isomerization or deterioration (U.S. Pat. No. 6,706,843, WO2021/126661). They are therefore not suitable for the depolymerization of certain PET-based materials, such as multifiber fabric materials, since they would lead to the production of PET molecules modified due to “pollution” by residues originating from the other constituents of the fabric. These modified molecules may be toxic or generate disruptions during the production of recycled PET, and are detrimental to the quality of the depolymerized product for these future applications.

Document WO2020/128218 describes a method for depolymerization of PET by alcoholysis using a monoalcohol such as methanol or ethanol and a base selected from sodium methoxide, KOH or NaOH in a stoichiometric amount relative to the PET.

It is known that the use of a base in catalytic amount relative to the mass of PET makes it possible to obtain DMT, but the kinetics of the reaction is quite slow, the reaction time is greater than 10 hours and 30 minutes, during which time the reaction solution is heated continuously. By way of example, mention may be made of US2019/0256450 and WO2020/188359, which describe the depolymerization of PET into DMT in the presence of methanol and an alkoxide such as sodium methoxide. These methanolysis reactions take place at temperatures of between 25° C. and 100° C. These methods necessarily comprise a first stage of swelling the PET with chlorinated solvents or polar solvents such as DMSO or DMF or methanol. US2019/0256450 proposes reacting the PET with a base, sodium methoxide in catalytic amount, and methanol. The method described in WO2020/188359 is characterized by the sequential addition of methanol and methylate solutions several times after addition of sodium methoxide. The authors describe high PET production yields. US2019/390035 describes another approach for depolymerization by adding glycolate salt, the preparation of this salt comprises isolation and drying steps that extend over one week.

WO2021/126661 describes an improved method for depolymerizing PET by methanolysis using catalysts selected from sodium carbonate, magnesium methoxide, DBU and TBD. This method is implemented at temperatures of at least 110-140° C. by applying a pressure of 15 bar.

For a person skilled in the art, the implementations of the methods described above obviously exhibit problems of industrial operability and feasibility regarding the safety aspect of an ATEX environment such as that of methanol under reflux, which requires complex precautions and expensive devices when introducing flammable products into the method.

None of these methods are satisfactory. It is therefore desirable to have improved methods for recycling PET-based materials, that are inexpensive and easily operable industrially to facilitate the generalization of this recycling and to broaden the scope of use of recycled PET, and more generally of terephthalate polyester.

### BRIEF SUMMARY

A novel method has been developed that is particularly efficient for depolymerization by alcoholysis under mild conditions for recycling materials comprising terephthalate polyester, in particular, polyethylene terephthalate (PET) or polybutylene terephthalate (PBT) into terephthalate ester monomers. This method is very fast while being much more environmentally friendly than the methods of the state of the art. It gives access to a product in solid form that is directly reusable due to its purity, in particular, DMT, DET or BHET in crystalline form.

Thus, the present disclosure relates to a method for recycling a material comprising a terephthalate polyester into a terephthalate ester comprising two steps:
a. a step of milling or shredding the material to produce fragments, and
b. a step of depolymerizing the terephthalate polyester into terephthalate ester, in the presence of:
   (i) a catalyst selected from a metal or organic ether oxide base, a metal acetate, a metal oxide, a metal hydroxide or a metal carbonate,
   (ii) a polar solvent of the cyclic ester or ether oxide type, and
   (iii) an alcohol selected from a monoalcohol or a diol
wherein:
   the base is present in catalytic amount relative to the amount of the terephthalate polyester
   the depolymerization step is carried out at ambient temperature or by heating up to 70° C. for a period of between 1 minute and 4 hours.

The terephthalate-polyester-based material may be a plastic, a fabric or another type of material containing 100% terephthalate polyester (PET or PBT) or a composite material containing a mixture of terephthalate polyester with other constituents such as cotton, polyamide, polyurethanes, polyolefins and fluorinated polymers, such as a composite plastic, a multifiber fabric or a composite insulation material.

The method according to the present disclosure proposes associating (i) a catalyst in catalytic amount relative to the terephthalate polyester, (ii) an alcohol that is either a monoalcohol or a diol, and (iii) a solvent of the ester type, and reacting them under mild conditions. It has several advantages relative to the methods described above, which are explained hereunder.

A first remarkable advantage: this method does not require pretreatment, a step which requires the use of toxic products. The depolymerization reaction is effective enough to allow complete depolymerization without prior swelling of the material to be treated. Thus, the method according to the present disclosure is simpler (one step less), more environmentally friendly (no toxic products and therefore no effluent to be treated), faster and less expensive.

Since the method entails a very moderate industrial risk, industrial installations for the implementation thereof can consequently be set up more easily, the level of security of these facilities being less constraining. Regulatory compliance is simplified during plant installation and throughout the production cycle. Capital expenditure is thus significantly reduced.

Any type of solvent can be used for depolymerization, although ester-type solvents are preferred. These products in fact are free of any toxicity and are used, in particular, in food processing, in the field of flavorings.

Remarkably, the depolymerization reaction is complete, very fast and produces a high purity terephthalate ester. This is applicable for PET and also for PBT, which are depolymerized into DMT, DET or BHET. The latter are then easily recyclable and have industrial outlets and a recognized market.

The method can be described as “very fast” since the reaction is complete in less than 4 hours at ambient temperature, and in less than 20 minutes under optimized heating conditions, in particular, between 55 and 70° C. It starts instantly and can lead to complete depolymerization within 1 min.

The depolymerization reaction is simple. Depolymerization and purification can be carried out in one and the same step. After completion of the reaction, the product obtained is directly a terephthalate ester in crystal form. Washing makes it possible to remove the intermediate or degradation products, which would require laborious distillation operations in the conventional methods to separate them from the product of interest.

The method thus makes it possible to obtain DMT, DET or BHET depending on whether the depolymerization of the PET or PBT is carried out by methanolysis, ethanolysis or glycolysis, respectively.

This method may be applied to any type of material comprising a terephthalate polyester, in particular, PET or PBT, pure or blended, transparent or colored, regardless of its thickness or its composition.

A person skilled in the art knows that composite fabric and multifiber materials can be manufactured in different ways. They may be woven and coated in the form of several layers, or of non-woven nature. They consist of different materials, in particular, polyester materials blended with other materials.

The nature of the “polyester” type materials may differ and, in non-exhaustive terms, the latter may be polyethylene terephthalate (PET); polybutylene terephthalate (PBT), polylactic acid (PLA), polycaprolactone (PCL), etc. The materials that can be recycled by virtue of the method according to the present disclosure comprise at least one terephthalic polyester of PET or PBT type.

The other materials blended with the polyester may be, in a non-limiting manner, polyamide (nylon 6,6 or hexamethylene diamine diadipate, nylon 6 or polycaprolactam, etc.), polyurethanes, cotton, polyolefins (polypropylene, polyethylene), fluorinated polymers (the latter are generally coated). Fluorinated polymers are used for their sealing and insulating properties; for example, polytetrafluoroethylene (PTFE). Among the polyurethanes, elastane is particularly appreciated because it provides flexibility and breathable properties to the fabric, in particular, when it is a flexible elastomer polyurethane, the two main commercially available forms of which are poly(ether)urethanes and poly(ester)urethanes.

The use of polyamides in variable proportions with PET has many applications in the field of clothing (lingerie and sports clothing) but also in technical fabrics for their highly absorbent and dust-free properties (microfiber wipes for industrial wiping). However, it is not currently possible to recycle a composite material comprising elastane, polyamides or coated materials, which is problematic in terms of waste management; the method according to the present disclosure provides a solution to this problem.

In addition, the method according to the present disclosure is particularly advantageous for recycling terephthalate-polyester-based composite material due to the fact that the reaction is selective relative to terephthalate polyester and does not modify the other optional components; the separation is thus easy between the depolymerized terephthalate polyester in the form of monomers and the other components; the latter may be recovered by simple filtration and washing. Next, after a possible bleaching step in the case of the fabric, the cooled mixture allows the terephthalate ester monomers to precipitate. Washing is sufficient to obtain DMT, DET or BHET that can be directly usable. The washing solvents are advantageously the alcohols used during depolymerization.

Another advantage is that it makes it possible to upcycle the separated materials of the terephthalate polyester after the depolymerization thereof, in an unaltered form. Polyamide (polyamide 6; polyamide 6,6), elastane and cotton are specific examples of this, in the field of fabrics. The method described herein enables isolation of the components initially blended with the terephthalate polyester with a degree of purity allowing for their subsequent recycling. It may be noted that one of the highly innovative applications of this method is to allow the recovery of elastane or polyamide from a composite material based on terephthalate polyester and the reuse thereof in new applications.

The yield of the method is high: at least 85%, in particular, for depolymerization of the PET into DMT. In addition, the material blended with the polyester is entirely restored.

In the particular case of the depolymerization of PET into DMT using methanol, the product obtained is 99.9% pure at the end of the reaction (after filtration and washing); there is therefore no need for subsequent purification. The DMT can be used directly after washing with methanol. Given its level of purity, it may be used in numerous applications, for remaking PET or any other type of technical resin involving this monomer. The choice of reagents and the implementation of mild conditions means that no isomerization reaction occurs, and that no degradation products that are detrimental to the quality of the product obtained are formed. When present, these secondary molecules to the reaction disrupt the polymerization reaction and a purification of the crude DMT is therefore necessary before it is used. This can be generalized to the depolymerization of the PET and PBT into terephthalate ester monomers of any type (DMT, DET and BHET).

This method is more economical and more environmentally friendly than the existing methods due to the fact that the bases (catalysts) are used in catalytic amounts relative to the amount of terephthalate polyester to be recycled, and that the reaction temperatures are lower than 80° C., generally between the ambient temperature (around 25° C.) and 60° C. and that the reaction times are greatly reduced compared to those of the PET depolymerization methods described in the literature.

In particular, the alcohol is used in proportions ranging from 0.25 to 16 molar equivalents relative to the terephthalate polyester; preferentially from 0.6 to 9 molar equivalents relative to the terephthalate polyester; more precisely from 1.1 to 4.9 molar equivalents relative to the terephthalate polyester, which is a substantial improvement relative to the conventional methanolysis technologies wherein proportions of 25 molar equivalents are necessary.

The proportions of the polar solvent of acetate type are also reduced ranging from the minimum of 1:1.5 for the terephthalate polyester mass:solvent mixture volume to a 1:10 ratio for the terephthalate polyester mass:solvent mixture volume.

From an ecological point of view, it should be noted that the depolymerization bath containing the solvent can be reused for a new treatment cycle once the product has been filtered. The bath can be used at least twice without affecting the effectiveness of the reaction. Once the reaction is finished, the solvents can be recovered by simple low-energy distillation, given their low boiling point.

### DETAILED DESCRIPTION

The present disclosure relates to a method for recycling a material comprising a terephthalate polyester into terephthalate ester comprising two steps:
a. a step of milling or shredding the waste to produce fragments, and
b. a step of depolymerizing the polyester into terephthalate ester in the presence of:
   (i) a catalyst selected from a metal or organic ether oxide base, a metal acetate, a metal oxide, a metal hydroxide, a metal carbonate or a metal ester,
   (ii) a polar solvent of the cyclic ester or ether oxide type, and
   (iii) an alcohol selected from a monoalcohol or a diol,
wherein:
   the base is present in catalytic amount relative to the amount of the polyester, and
   the depolymerization step is carried out at ambient temperature or by heating up to 70° C. for a period of between 1 minute and 4 hours.

In a preferred embodiment, the present disclosure relates to a method for recycling materials comprising polyethylene terephthalate into terephthalate ester monomers comprising two steps:
a. a step of milling or shredding the waste to produce fragments, and
b. a step of depolymerizing the PET into terephthalate ester and monoethylene glycol (MEG), in the presence of:
   (i) a catalyst selected from a metal or organic ether oxide base, a metal acetate, a metal oxide, a metal hydroxide, a metal carbonate or a metal ester,
   (ii) a polar solvent of the cyclic ester or ether oxide type, and
   (iii) an alcohol selected from a monoalcohol or a diol,
wherein:
   the base is present in catalytic amount relative to the amount of PET, and
   the depolymerization step is carried out at ambient temperature or by heating up to 70° C. for a period of between 1 minute and 4 hours.

It is known that the depolymerization of a terephthalate polyester produces DMT and a diol corresponding to the polyester, namely monoethylene glycol from PET and butane diol from PBT.

Materials comprising terephthalate polyester may consist of 100% terephthalate polyester (for example, plastics or fabrics) or may consists of a mixture comprising terephthalate polyester and other components such as cotton, polyamide, elastane, PTFE, polyethylene, polypropylene (for example, composite plastics, multifiber fabrics or insulating composite panels).

When the material comprising terephthalate polyester consists of 100% terephthalate polyester, this material is transformed into terephthalate ester. This can be recovered by simple filtration and precipitated by cooling, as described hereunder and illustrated in the experimental section.

The terephthalate polyester is selected from polyethylene terephthalate (PET) and polybutylene terephthalate (PBT).

When the material comprising terephthalate polyester is a composite material comprising terephthalate polyester blended with other components, the recycling method produces terephthalate esters (DMT) by depolymerization of the terephthalate polyester, the other components of the material being in unaltered form in the reaction mixture. Since these other components are larger than the DMT, they can be separated from the latter by simple filtration.

The composite material comprises terephthalate polyester selected from polyethylene terephthalate and polybutylene terephthalate, blended with another component selected from cotton, polyamide, polyurethanes, polyolefins and fluorinated polymers.

Thus, in a particular embodiment wherein the material comprising the PET is a composite material comprising PET blended with other components, the recycling method makes it possible to transform (recycling) the PET into DMT and to release the other components (which may also be recycled).

The catalyst is a base selected from:
(i) an ether oxide is of the sodium methoxide, magnesium methoxide, potassium methoxide or ammonium methoxide type,
(ii) a metal carbonate of sodium carbonate or potassium carbonate type,
(iii) a metal hydroxide of sodium hydroxide or potassium hydroxide type,
(iv) a metal acetate of zinc acetate Zn(OAc)₂ or sodium acetate NaOAc type, or a potassium acetate KOAc,
(v) a metal oxide,
(vi) a metal ester of titanium ester Ti(OiPr)₄, manganese ester Mn(OR)₂, or antimony ester Sb(OR)₂ type.

In a preferred embodiment of the present disclosure, the catalyst is selected from sodium methoxide, magnesium methoxide, potassium methoxide or ammonium methoxide.

The catalyst is present in a molar ratio of less than 35% relative to the terephthalate polyester, preferably between 1% and 20%.

The ester-type solvent may be a monoester, a diester or a triester.

The ester-type solvent preferably corresponds to molecular formula A:

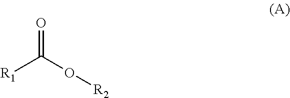

The ether oxide-type solvent preferably corresponds to molecular formula B:

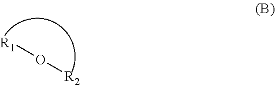

wherein R₁ and R₂ are identical or different and are selected (independently) from an aryl CₙH₂ₙ, alkyl CₙH₂ₙ₊₁ or CₙH₂ₙ₋₁ with n=1 to 10.

In a preferred embodiment, the polar solvent is of ester type since it is non-toxic. The ester-type solvent can be selected from methyl acetate, ethyl acetate, propyl, butyl, or isopropyl.

Table 2 (experimental part) describes different embodiments of the present disclosure depending on the base used.

The solvent may also be of the cyclic ether oxide type, such as dioxane.

In a preferred embodiment of the present disclosure, the terephthalate polyester: solvent ratio is between 1:1.5 and 1:10.

The amount of alcohol involved in the depolymerization reaction is variable. The alcohol can be either supplied by the base in solution (in an alcohol), or added as such in the reaction medium. The alcohol can thus be in excess, in equivalent amount or deficient relative to the amount of terephthalate polyester. This parameter will be adjusted by a person skilled in the art.

The alcohol is present in a ratio of between 0.25 and 16 molar equivalents relative to the terephthalate polyester. In another preferred embodiment of the present disclosure, the alcohol:terephthalate polyester molar ratio is between 0.5 and 16; preferably between 0.6 and 9; more preferably between 1.1 and 3.

Advantageously, the method is implemented by applying a terephthalate polyester: solvent mixture ratio of between 1:1.5 and 1:10 and an alcohol:terephthalate polyester molar ratio of between 0.25 and 10. In a particular embodiment, the method is implemented by applying a terephthalate polyester: solvent mixture ratio of between 1:1.5 and 1:5 and an alcohol:terephthalate polyester molar ratio of between 0.25 and 3.

The alcohol used during the depolymerization step is preferably a monoalcohol selected from methanol, ethanol, propanol or butanol, or a diol such as ethylene glycol.

In a particular embodiment of the present disclosure, an alcohol and an ester of the same rank are used during the depolymerization reaction.

This combination of an alcohol and an ester of the same rank has the advantage of allowing a complete depolymerization reaction. The terephthalate monomers are thus solubilized. It suffices to cool the solution to precipitate them and recover a high-purity product (at least 99%).

If the material includes a blend of terephthalate polyester and other components, the latter will not be modified, will remain in suspension and will be easily removed by filtration.

By way of example, the methanol and the methyl acetate can be combined, DMT (methanolysis reaction) or ethanol and ethyl acetate are obtained, DET (diethyl terephthalate diester) is obtained (ethanolysis reaction). If diethylene glycol is used, BHET (bis(2-Hydroxyethyl) terephthalate) is obtained (glycolysis reaction).

The interest of DET is, for example, illustrated in document WO2007/076384, which describes an ethanolysis reaction of PET. DET production is described as advantageous the fact that DET is easier to dissolve than DMT. The DET obtained can be oxidized and then used to produce terephthalic acid.

Alternatively, another embodiment according to the present disclosure may involve combining an alcohol and an ester of different rank. For example, ethyl acetate and methanol, two commonly used reagents, can be combined. The PET depolymerization reaction is carried out effectively and completely and a majority product corresponding to the alcohol used is obtained, in this example DMT due to the presence of methanol, but also secondary products such as DET and other terephthalate monomers.

The base involved in the depolymerization reaction may be in catalytic amount relative to the amount of terephthalate polyester to be treated.

“Catalytic amount” means a non-stoichiometric amount, that is to say, in a molar ratio of 1% to 49% relative to the amount of terephthalate polyester to be treated. The term “catalytic” also applies to a reagent that is found in its initial form at the end of the reaction (catalyst).

In a preferred embodiment of the present disclosure, the catalytic amount of ether oxide base is less than 35 mol %. The catalytic amount of ether oxide base may vary from 1 mol % to 35 mol %, preferably from 1 mol % to 20 mol %, or even from 5 mol % to 20 mol %. Prolonged reaction times can be applied to further reduce this amount, which makes it possible to reduce the cost of the reaction.

The reaction temperature may vary. The reaction medium can be heated to 70° C. The mixture can, in particular, be advantageously heated between 50° C. and 70° C., preferably to a temperature below 60° C. However, it is very interesting to note that the reaction operates very well at room temperature (around 25° C.) while being fast, since complete depolymerization is obtained in 3 to 4 hours. The fact of not heating the reaction simplifies the implementation and reduces the cost.

The present disclosure will be better understood upon reading the examples that follow, provided by way of illustration, and in no way considered to be limiting on the scope of the invention.

### EXPERIMENTAL PART

### Example 1: PET Depolymerization by Methanolysis

An amount (500 g) of polyethylene terephthalate (PET) pieces originating from different sources (food trays, water bottles, etc.) is introduced into 2 L of methyl acetate. 120 mL of a sodium methoxide solution (25% in methanol) corresponding to a molar ratio of 20% sodium methoxide relative to the PET introduced and 200 mL of methanol are added to the pieces. The reaction begins instantaneously. After 30 minutes of reaction at 55° C., all the pieces of PET have disappeared leaving a white solid slightly suspended in solution. The crude reaction mixture is filtered through a Büchner filter in order to retain the unreacted material; the recovered medium gels almost instantaneously. It contains the DMT, the monoethylene glycol produced by the depolymerization reaction as well as the initially reacted base and the solvent. The white solid (DMT) is recovered (410 g, 82%) and is washed with methanol.

### Example 2: PET Depolymerization by Methanolysis

An amount (500 g) of PET pieces originating from different sources (food trays, water bottles, etc.) is introduced into 2 L of methyl acetate. 210 mL of a sodium methoxide solution (25% in methanol) corresponding to a molar ratio of 35% sodium methoxide relative to the PET introduced are added to the pieces. The reaction begins instantaneously. After 30 minutes of reaction at 55° C., all the pieces of PET have disappeared leaving a white solid slightly suspended in solution. The crude reaction mixture is filtered through a Büchner filter in order to retain the unreacted material; the recovered medium gels almost instantaneously. It contains the DMT, the monoethylene glycol produced by the depolymerization reaction as well as the initially reacted base and the solvent. The white solid (DMT) is recovered (400 g, 80%) and is washed with methanol.

### Example 3: Depolymerization of 100% PET Fabric by Methanolysis

An amount (500 g) of 100% PET colored fabric pieces is introduced into 2 L of methyl acetate. 119 mL of a sodium methoxide solution (25% in methanol) corresponding to a molar ratio of 20% sodium methoxide relative to the PET introduced and are added to the pieces. The reaction begins instantaneously. After 120 minutes of reaction at 55° C., all the pieces of PET are depolymerized, leaving a colored solid slightly suspended in solution. A bleaching step was carried out by adding activated carbon in order to obtain a white DMT. The crude reaction mixture is filtered through a Büchner filter in order to retain the unreacted material and the activated carbon; the recovered medium gels almost instantaneously. It contains the DMT, the monoethylene glycol produced by the depolymerization reaction as well as the initially reacted base and the solvent. The white solid (DMT) is recovered (350 g, 70%) and is washed with methanol.

### Example 4: Depolymerization of an 85%/15% PET/Elastane Blended Fabric Material by Methanolysis

An amount (500 g) of pieces of PET/elastane blended fabric composed of 85% colored PET and 15% colored elastane containing different percentages of elastane was introduced into 3.2 L of methyl acetate and 0.8 L of methanol. 119 mL of a sodium methoxide solution (25% in methanol) corresponding to a molar ratio of 20% sodium methoxide relative to the fabric introduced are added to the pieces. The reaction begins instantaneously. After 80 minutes of reaction at 55° C., all the PET-based fabric pieces were depolymerized leaving the unreacted elastane pieces and a colored solid slightly suspended in solution. A prefiltration step was carried out to retain the elastane (75 g, 15%) and the unreacted material. The next step is bleaching by adding the activated carbon to the crude reaction mixture in order to obtain a white DMT. The crude reaction mixture is filtered through a Büchner filter in order to retain the activated carbon; the recovered medium gels almost instantaneously. It contains the DMT, the monoethylene glycol produced by the depolymerization reaction as well as the initially reacted base and the solvent. The white solid (DMT) is recovered (280 g, 66%) and is washed with methanol.

### Example 5: Depolymerization of the PET/Cotton (80%/20%) Blended Fabric by Methanolysis

An amount (500 g) of pieces of PET/cotton blended fabric composed of 80% colored PET and 20% colored cotton is introduced into 5 L of methyl acetate. 119 mL of a sodium methoxide solution (25% in methanol) corresponding to a molar ratio of 20% sodium methoxide relative to the fabric introduced are added to the pieces. The reaction begins instantaneously. After 210 minutes of reaction at 55° C., the majority of the pieces of PET fabric have deteriorated leaving the unreacted pieces of cotton and a colored solid slightly suspended in solution. A prefiltration step was carried out to retain the cotton (165 g, 20%) and the unreacted material. The next step is bleaching by adding the activated carbon to the crude reaction mixture in order to obtain a white DMT. The crude reaction mixture is filtered through a Büchner filter in order to retain the activated carbon; the recovered medium gels almost instantaneously. It contains the DMT, the monoethylene glycol produced by the depolymerization reaction as well as the initially reacted base and the solvent. The white solid (DMT) is recovered (245 g, 61%) and is washed with methanol.

### Example 6: Depolymerization of the PET/Polyamide (90%/10%) Blended Fabric by Methanolysis

An amount (500 g) of colored PET/PA blended fabric pieces is introduced into 5 L of methyl acetate. 119 mL of a sodium methoxide solution (25% in methanol) corresponding to a molar ratio of 20% sodium methoxide relative to the fabric introduced are added to the pieces. The reaction begins instantaneously. After 120 minutes of reaction at 55° C., all the pieces of fabric have degraded, leaving the pieces of unreacted PA and a colored solid slightly suspended in solution. A prefiltration step was carried out to retain the PA (50 g, 10%) and the unreacted material. The next step is bleaching by adding the activated carbon to the crude reaction mixture in order to obtain a white DMT. The crude reaction mixture is filtered through a Büchner filter in order to retain the activated carbon; the recovered medium gels almost instantaneously. It contains the DMT, the monoethylene glycol produced by the depolymerization reaction as well as the initially reacted base and the solvent. The white solid (DMT) is recovered (350 g, 70%) and is washed with methanol.

### Example 7: Depolymerization of the 100% PET Composite Insulation Panel by Methanolysis

An amount (500 g) of white pieces of 100% PET foam is introduced into 1.5 L of methyl acetate. 71.43 mL of a sodium methoxide solution (25% in methanol) corresponding to a molar ratio of 12% sodium methoxide relative to the PET introduced are added to the pieces. The reaction begins instantaneously. After 20 minutes of reaction at 55° C., all the pieces of insulation panel have degraded leaving a light yellow solid slightly suspended in solution. The crude reaction mixture is filtered through a Büchner filter in order to retain the unreacted material; the recovered medium gels almost instantaneously. It contains the DMT, the monoethylene glycol produced by the depolymerization reaction as well as the initially reacted base and the solvent. The white solid (DMT) is recovered (360 g, 72%) and is washed with methanol.

### Example 8: PET Depolymerization by Ethanolysis

An amount (500 g) of PET pieces originating from different sources (food trays, water bottles, etc.) is introduced into 2 L of ethyl acetate. 28.12 g of sodium methoxide corresponding to a molar ratio of 20% sodium methoxide relative to the PET introduced and 300 mL of ethanol are added to the pieces. The reaction begins instantaneously. After 30 minutes of reaction at 70° C., all the pieces of PET have disappeared leaving a white solid slightly suspended in solution. The crude reaction mixture is filtered through a Büchner filter in order to retain the unreacted material, the recovered medium contains the DET, the monoethylene glycol produced by the depolymerization reaction as well as the base initially reacted and the solvent. The DET (400 g) is recovered in the form of a pasty solid following the evaporation of the reaction solvents and is washed with ethanol.

### Example 9: Conversion Rate as a Function of Time and Temperature

Table 1 shows the effect of the reaction time and temperature on the conversion rate of PET to DMT.

The following reaction conditions are used: 10 g of PET are incubated in a sodium methoxide solution (diluted to 25% in MeOH) in a ratio of 20% (mole:mole of PET), in the presence of 45 ml of methyl acetate.

TABLE 1
Effect of time/temperature on the conversion rate

| | Time | | |
|---|---|---|---|
| Temperature | 30 minutes | 60 minutes | 180 minutes |
| 25° C. (AT) | 64% | — | 77% |
| 35° C. | — | 78% | 98% |
| 60° C. | 99% | 99.7% | — |

### Example 10: Conversion Rate as a Function of the Type of Solvent and Alcohol

Table 2 shows the effect of the reaction time and temperature on the conversion rate of PET to DMT.

The reaction conditions are the same as those of Example 4.

The ester-type solvent has the formula hereunder:

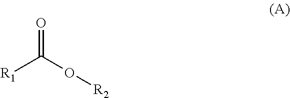

TABLE 2
Effect of varying the solvent/alcohol type on the conversion rate

| | R1/R2 | | | | | |
|---|---|---|---|---|---|---|
| % Base | CH₃/CH₃ | CH₃/C₂H₅ | nC₃H₇/CH₃ | C₄H₉/CH₃ | C₄H₉/C₂H₃ | CH₃/ᵢₛₒprop |
| 7.5% | 97% | — | — | — | — | — |
| 10% | — | 94% | — | — | — | — |
| 15% | 99% | 99% | — | — | — | — |
| 20% | 99% | 99% | 95% | 93% | — | — |
| 35% | 99% | 99% | — | — | 85% | 99% |

1. A method for recycling a material comprising terephthalate polyester into terephthalate diester, comprising:
a. milling or shredding the material to produce fragments; and
b. a step of depolymerizing the polyester into terephthalate ester in the presence of:
(i) a catalyst selected from a metal or organic ether oxide base, a metal acetate, a metal oxide, a metal hydroxide, a metal ester or a metal carbonate,
(ii) a polar solvent of the cyclic ester or ether oxide type,
(iii) an alcohol selected from a monoalcohol or a diol,
wherein:
the catalyst is present in catalytic amount relative to the amount of the polyester, and
the depolymerization step is carried out at ambient temperature or by heating up to 70° C. for a period of between 1 minute and 4 hours.

2. The method of claim 1, wherein the material is composed of 100% terephthalate polyester selected from polyethylene terephthalate (PET) and polybutylene terephthalate (PBT).

3. The method of claim 1, wherein the material is a composite material comprising terephthalate polyester selected from polyethylene terephthalate and polybutylene terephthalate blended with another component selected from cotton, polyamide, polyurethanes, polyolefins and fluorinated polymers.

4. The method of claim 3, wherein the polyurethane is elastane.

5. The method of claim 4, wherein the catalyst is selected from among (i) sodium methoxide, potassium methoxide, magnesium methoxide or ammonium methoxide; (ii) sodium carbonate or potassium carbonate; (iii) sodium hydroxide or potassium hydroxide; (iv) zinc acetate or sodium acetate or potassium acetate; (v) a metal oxide; (vi) a metal ester; or (vii) titanium ester.

6. The method of claim 5, wherein the catalyst is a metal ester selected from titanium ester, manganese ester, antimony ester, or zinc acetate, sodium acetate or potassium acetate.

7. The method of claim 6, wherein the catalyst is present in a molar ratio of less than 35% relative to the terephthalate polyester.

8. The method of claim 1, wherein the polar solvent corresponds to one of molecular formulas A or B: 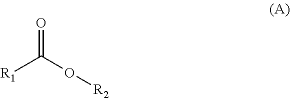 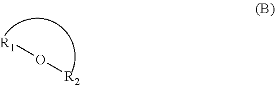
wherein R₁ and R₂ are identical or different and are selected from an aryl CₙH₂ₙ, alkyl CₙH₂ₙ₊₁ or CₙH₂ₙ₋₁ with n=1 to 10.

9. The method of claim 8, wherein the ester is selected from methyl acetate, ethyl acetate, propyl, butyl, or isopropyl.

10. The method of claim 1, wherein the terephthalate polyester: solvent ratio is between 1:1.5 and 1:10.

11. The method of claim 1, wherein the alcohol is selected from methanol, ethanol, propanol or butanol.

12. The method of claim 1, wherein the alcohol:terephthalate polyester molar ratio (equivalent) is comprised between 0.25 and 16.

13. The method of claim 1, wherein an alcohol and an ester of the same rank are used for the same depolymerization reaction.

14. The method of claim 1, wherein the alcohol is ethylene glycol.

15. The method of claim 1, wherein the catalyst is selected from among (i) sodium methoxide, potassium methoxide, magnesium methoxide or ammonium methoxide; (ii) sodium carbonate or potassium carbonate; (iii) sodium hydroxide or potassium hydroxide; (iv) zinc acetate or sodium acetate or potassium acetate; (v) a metal oxide; (vi) a metal ester; or (vii) titanium ester.

16. The method of claim 15, wherein the catalyst is a metal ester selected from titanium ester, manganese ester, antimony ester, or zinc acetate, sodium acetate or potassium acetate.

17. The method of claim 16, wherein the catalyst is present in a molar ratio of less than 35% relative to the terephthalate polyester.

18. The method of claim 1, wherein the catalyst is selected from among sodium methoxide, potassium methoxide, magnesium methoxide or ammonium methoxide.

19. The method of claim 1, wherein the catalyst is selected from among sodium carbonate or potassium carbonate.

20. The method of claim 1, wherein the catalyst is selected from among sodium hydroxide or potassium hydroxide.
